(19) United States
(12) Patent Application Publication
(10) Pub. No.: US 2024/0399166 A1
(43) Pub. Date: Dec. 5, 2024

(54) Method For Reducing Visceral Body Fat In Humans

(71) Applicant: Doug Zucco, Las Vegas, NV (US)

(72) Inventor: Doug Zucco, Las Vegas, NV (US)

(21) Appl. No.: 18/800,123

(22) Filed: Aug. 11, 2024

Related U.S. Application Data
(63) Continuation-in-part of application No. 16/870,846, filed on May 8, 2020, now abandoned.

(51) Int. Cl. A61N 5/06; A61H 1/00; A61N 2/00

(52) U.S. Cl. CPC A61N 5/0616; A61H 1/005; A61N 2/002; A61N 2005/0659; A61N 2005/0662

(57) ABSTRACT

A method for reducing visceral body fat in humans involving the steps of a user utilizing a pulsed electromagnetic field generating device for a recommended period of time; the user then laying in an LED therapy bed with a light chamber having an LED array configured to emit multiple wavelengths of light at differing output percentages, the LED array of the LED therapy bed producing a chamber spectrum combination of at least three different light wavelengths for a preferred length of time; and the user then utilizing a vibration platform for a preferred length of time. The method produces a synergistic effect, and may be achieved through the use of custom designed equipment and methods.

### CROSS-REFERENCE TO RELATED APPLICATIONS

The present utility patent application claims priority benefit of the U.S. provisional application for patent Ser. No. 62/845,756 titled “Process for Fat Loss Using a Laser Device and Related Methods,” filed on May 9, 2019 under 35 U.S.C. 119(e). The contents of this related provisional application are incorporated herein by reference for all purposes to the extent that such subject matter is not inconsistent herewith or limiting hereof.

### RELATED CO-PENDING U.S. PATENT APPLICATIONS

Continuation-in-part of U.S. nonprovisional application Ser. No. 16/870,846 filed on May 8, 2020.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office, patent file or records, but otherwise reserves all copyright rights whatsoever.

### BACKGROUND OF THE INVENTION

### 1. Field of the Invention

The invention relates generally to weight loss. More particularly, the invention is directed towards a method or process for visceral fat loss in humans using an LED light therapy bed and methods related thereto.

### 2. Description of the Related Art

Many people today suffer from excess body fat. Many different fat loss strategies and plans can be used to improve health and create a better physique.

A form of abdominal fat, called visceral fat or visceral body fat, can considerably increase risk for diabetes, heart disease and a number of other ailments. Unlike subcutaneous fat, visceral fat is body fat that is stored within the abdominal cavity and is therefore stored around a number of important internal organs such as the liver, pancreas and intestines. Subcutaneous fat is located superior, or above, the abdominal muscles; and is much easier to remove by known methods. Visceral fat is located inferior or beneath the abdominal muscles, and is much more difficult to target and reduce by known methods.

Visceral fat levels are considered by some experts to be more important and critical to overall wellness, and to long-term weight loss by keeping the unhealthy fat cells from returning. It is therefore important to implement new systems and methods for the reduction of visceral fat in humans.

Traditional methods for losing visceral fat involve modifications to diet along with increased physical activity. Many people, however, fail to achieve weight loss results through such traditional methods.

Other methods for losing visceral fat involve pharmacotherapy. Numerous drugs have been created to increase metabolism or to inhibit certain catabolic pathways preventing absorption and metabolism of fats and sugars. For many people, though, drug therapies may have dangerous or uncomfortable side effects which make such therapies undesirable.

Some suffering from excess fat can opt to have fat removed surgically through a procedure commonly known as liposuction. Liposuction removes fat cells from the body but cannot remove visceral fat from the body. Many users, though, would prefer a noninvasive form of treatment.

A new form of noninvasive weight loss therapy is the use of infrared light. Infrared light therapy uses near infrared light, usually from devices such as, but not limited to, light emitting diodes, lasers, lamps, or tanning bed-like devices. In such a therapy, patient is told to either lie down or sit in front of the light for a specific amount of time on a regular basis (usually once a day). Another popular therapy is the use of an infrared body wrap, consisting of large silicone bandages or pads that emit infrared light around the legs, torso and arms. Low level laser therapy is another form of infrared light therapy currently in development. Low level laser therapy involves the use specific wavelengths of red and infrared light to a user. At specific frequencies, wavelengths, coherence and power settings, fat cells absorb the electromagnetic energy from the light. When enough energy is absorbed, transitory (short-lived) pores open on the surface of fat cells. Water, glycerol and free fatty acids are broken down and released through the body's catabolic and metabolic pathways.

The use of infrared radiation to release fat has limitations. It takes time for infrared radiation to reach visceral body fat. Furthermore, when fat is released by infrared radiation, the body must have efficient ways to metabolize or clear such fat without reabsorbing it elsewhere.

It is therefore desired to create an improved system and method for visceral fat loss in humans using infrared and near-infrared light therapy techniques. Presently, there exists a need for a method or process for the reduction of visceral body fat which utilizes the benefits of devices designed to increase blood flow in conjunction with targeted infrared and red-light therapy to help reduce visceral body fat.

### SUMMARY

The object of the present invention is to provide an improved system, process or method for reducing visceral body fat in humans which utilizes the benefits of increased blood flow, increased muscular stimulation, as well as targeted infrared and red-light therapy to help reduce visceral body fat in a human user.

At its essence, the method for reducing visceral body fat involves the steps of a user utilizing a pulsed electromagnetic field generating device for a recommended period of time; the user then laying in an LED therapy bed with a light chamber having an LED array configured to emit multiple wavelengths of light at differing output percentages, the LED array of the LED therapy bed producing a chamber spectrum combination of at least three different light wavelengths for a preferred length of time; and the user then utilizing a vibration platform for a preferred length of time.

The method or process may be achieved through the use of custom designed equipment and methods. In embodiments of the invention, a pulsed electromagnetic force device is used to increase blood flow throughout the body. A light therapy bed is used to irradiate a user with specific wavelengths of red and infrared radiation. Finally, a vibration device is used to stimulate muscle contraction and movement throughout the body. Such a method or process has produced a synergistic effect.

By utilizing a body composition analyzer, both expected and unexpected results have been achieved. Used as recommended, the method for reducing visceral body fat in humans has show to not only lead to weight loss, but also desirable body sculpting results as a result of the targeting of deep visceral fat which is not normally metabolized absent novel methods.

### BRIEF DESCRIPTION OF THE DRAWINGS

The invention directed by way of example, and not by way of limitation, in the figures of the accompanying drawings and in which like reference numerals refer to similar elements and in which:

FIG. 1 illustrates a perspective view of a user laying on a pulsed electromagnetic field (PEMF) device;

FIG. 2 illustrates a perspective view of a user laying in a LED light therapy bed emitting infrared and red light;

FIG. 3 illustrates a perspective view of an LED light therapy bed and a fat loss chamber spectrum chart in accordance with an embodiment of the invention;

FIG. 4 illustrates a target light spectrum specifically targeted for the reduction of visceral fat; and

FIG. 5 illustrates a user standing on a vibration platform designed to facilitate increased blood flow

Unless otherwise indicated illustrations in the figures are not necessarily drawn to scale.

### DETAILED DESCRIPTION OF THE PREFERRED EMBODIMENTS

Terminology used herein is used for the purpose of describing particular embodiments only, and is not intended to limit the scope of the present invention. It must be understood that as used herein and in the appended claims, the singular forms “a,” “an,” and “the” include the plural reference unless the context clearly dictates otherwise. For example, a reference to “an element” is a reference to one or more elements and includes all equivalents known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Language that may be construed to express approximation should be so understood unless the context clearly dictates otherwise.

Unless defined otherwise, all technical and scientific terms used herein have the same meanings as commonly understood by a person of ordinary skill in the art to which this invention belongs. Preferred methods, techniques, devices, and materials are described. But any methods, techniques, devices, or materials similar or equivalent to those described herein may be used in the practice or testing of the present invention. Structures described herein should also be understood to refer to functional equivalents of such structures.

References to “one embodiment,” “an embodiment,” “various embodiments,” etc., may indicate that the embodiment(s) of the invention so described may include particular features, structures, or characteristics. However, not every embodiment necessarily includes the particular features, structures, or characteristics. Further, repeated use of the phrase “in one embodiment,” or “in an exemplary embodiment,” do not necessarily refer to the same embodiment although they may. A description of an embodiment with several components in communication with each other does not imply that all such components are required. On the contrary, a variety of optional components are described to illustrate the wide variety of possible embodiments of the present invention.

As is well known to those skilled in the art, many careful considerations and compromises typically must be made when designing for the optimal manufacture of a commercial implementation of such a method for reducing visceral body fat in humans. A commercial implementation in accordance with the spirit and teachings of the invention may be configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art.

The example embodiments described herein can be implemented in an operating environment comprising computer-executable instructions (e.g., software or firmware) installed on a computer, in hardware, or in a combination of software and hardware. The computer-executable instructions can be written in a computer programming language or can be embodied in firmware logic. If written in a programming language conforming to a recognized standard, such instructions can be executed on a variety of hardware platforms and for interfaces to a variety of operating systems. Although not limited thereto, computer software program code for carrying out operations for aspects of the present invention can be written in any combination of one or more suitable programming languages, including an object oriented programming languages and/or conventional procedural programming languages, and/or programming languages such as, for example, Hypertext Markup Language (HTML), Dynamic HTML, Extensible Markup Language (XML), Extensible Stylesheet Language (XSL), Document Style Semantics and Specification Language (DSSSL), Cascading Style Sheets (CSS), Synchronized Multimedia Integration Language (SMIL), Wireless Markup Language (WML), Java™, Jini™, C, C++, Smalltalk, Perl, UNIX Shell, Visual Basic or Visual Basic Script, Virtual Reality Markup Language (VRML), ColdFusion™, SQL, Python, or other compilers, assemblers, interpreters or other computer languages or platforms.

A light-emitting diode (LED) is a semiconductor light source that emits light when current flows through it. In a LED, electrons in the semiconductor recombine with electron holes, releasing energy in the form of photons. This effect is called electroluminescence. The color of the light (corresponding to the energy of the photons) is determined by the energy required for electrons to cross the band gap of the semiconductor. White light may be obtained by using multiple semiconductors or a layer of light-emitting phosphor on the semiconductor device. LEDs have many advantages over incandescent light sources, including lower energy consumption, longer lifetime, improved physical robustness, smaller size, and faster switching. Light-emitting diodes are used in a growing number of applications and may be used to transmit coherent light of specific wavelengths.

Embodiments of the method for reducing visceral body fat in humans may include more than one device for performing the operations disclosed herein. An apparatus or device may be specially constructed for the desired purposes, or it may comprise one or more general-purpose devices selectively activated or reconfigured by a program stored in the device. Moreover, embodiments of the method for reducing visceral body fat in humans may employ differing shapes and sizes to achieve a customized look.

Embodiments of the method for reducing visceral body fat in humans may also be implemented in, or in a combination of, hardware, firmware, and software. Certain embodiments may be implemented as instructions stored on a machine-readable medium, which may be read and executed by one or more specialty devices to perform operations to be carried out by the invention.

Unless specifically stated otherwise, and as may be apparent from the following description and claims, it should be understood that throughout this application's specification, descriptions utilizing terms such as “processing,” “computing,” “calculating,” “determining,” or the like, refer to the actions and/or processes of a computer, computing system, or any similar electronic computing device which manipulates and/or transforms data represented as physical quantities within the computing system's registers and/or memories into other data similarly represented as physical quantities within the computing system's memories, registers or other such information storage, transmission or display devices.

Aspects of the exemplary method for reducing visceral body fat in humans will be described below with reference to flowchart illustrations, tables, and/or block diagrams of methods, steps, apparatus (systems) and computer program products according to embodiments of the invention. Persons skilled in the art will understand that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

When a single device or article is described herein, it will be readily apparent to persons having skill in the art that more than one device or article or/machine (whether or not they cooperate) may be used in place of a single device or article or machine. Similarly, where more than one device or article or machine is described herein (whether or not they cooperate), it will be readily apparent that a single device/article may be used in place of the more than one device or article. Likewise, the functionality and/or the features of a device or article or machine may be alternatively embodied by one or more other devices or articles or machines which are not explicitly described as having such functionality and/or features. Thus, other embodiments of the present invention need not include a specific device in and of itself.

As is well known to those skilled in the art, many careful considerations and compromises typically must be made when designing the optimal manufacture or commercial implementation of such a method for reducing visceral body fat in humans. A commercial implementation in accordance with the spirit and teachings of the invention may be configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art.

The exemplary method for reducing visceral body fat in humans will now be described in detail with reference to embodiments thereof as illustrated in the accompanying drawings.

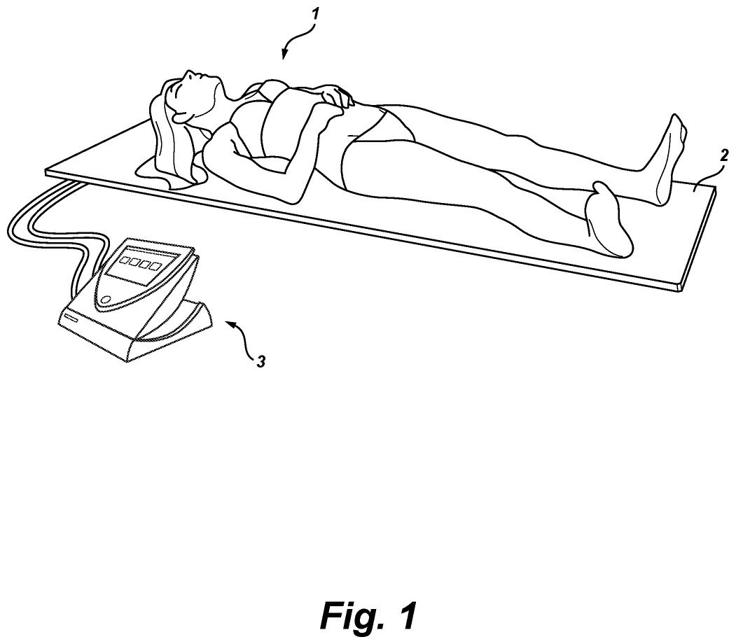

FIG. 1 illustrates a perspective view of a user 1 laying on a pulsed electromagnetic field (PEMF) device. The majority of PEMF wellness devices resemble a typical mat in dimensions but are slightly thicker to house several flat spiral coils to produce an even electromagnetic field. A frequency generator is then used to energize the coils to create a pulsed electromagnetic field. For therapeutic use, PEMF devices are typically placed on a massage table for clinical use. Persons having skill in the art will recognize that a PEMF device consists generally of a pulsed electromagnetic field machine 2 and a pulsed electromagnetic field plate 3. One such PEMF device is commonly referred to as a BEMER™ device manufactured by the BEMER Group. The components of such devices function to generate a pulsed electromagnetic field, wherein pulse sequences with defined levels, defined intervals and defined frequencies influence the pulsation of the electromagnetic field. The PEMF device generates a relatively weak magnetic field while transmitting electromagnetic pulses of varying frequencies and wavelengths.

Persons skilled in the art will readily appreciate that pulsed electromagnetic field therapy has been shown to increase blood flow throughout a human body. Persons having skill in the art will further appreciate that PEMF therapy has been shown to influence cellular behavior by inducing electrical changes around and within cells. Various clinical studies have shown PEMF therapy to improve blood supply and cause an increase in oxygen pressure, activating and regenerating cells.

Several studies have reported that PEMF can modulate both cell surface receptor expression/activation and downstream signal transduction pathways, thereby restoring homeostatic cell functions such as viability, proliferation, differentiation, communication with neighboring cells, and interaction with components of the extracellular matrix (ECM). PEMF can activate multiple intracellular pathways, including numerous processes and biochemical mechanisms within both the immune system and tissue regenerative processes, such as the musculoskeletal system and the nervous system. Pulsed electromagnetic fields can further serve as physical stimuli that affect biological systems through the production of coherent or interfering fields that modify fundamental electromagnetic frequencies generated by living organisms.

The object of using a pulsed electromagnetic field generating device is to both increase blood flow throughout the body for the purpose of preparing the body for red and infrared light therapy and to activate multiple intracellular pathways such as increasing cell porosity for the purpose of lipolysis. Such improvements in circulation and microcirculation offers a large range of positive benefits including transportation of oxygen and nutrients.

In an embodiment of the invention, a user lays for 8-20 minutes on a pulsed electromagnetic field generating plate. Persons skilled in the art will understand that differing lengths of time along with differing magnetic field strengths and electromagnetic radiation frequencies and wavelengths may be employed as well. It will also be readily apparent to persons having skill in the art that a wide variety of pulsed electromagnetic field devices exist, and that a user need not lay on a device to utilize and obtain the benefits of such PEMF therapy.

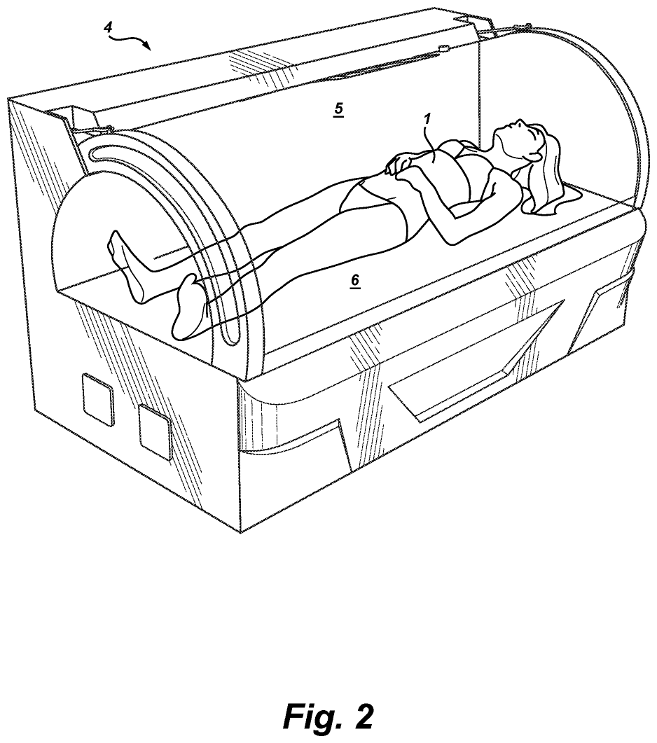

FIG. 2 illustrates a perspective view of a user 1 laying in a LED light therapy bed 4 emitting infrared and red light. Upon exposure to certain wavelengths of light, transitory pores open on the surface of visceral fat cells. This opening of pores allows for the release of free fatty acids and triglycerides which are then broken down, reused, and released through the body's catabolic metabolic pathways. Near infrared and red light therapy uses a low-powered red light to deliver specific wavelengths of electromagnetic radiation directly through the skin, which can reach deep visceral fat cells beneath subcutaneous fat as well as abdominal muscle. This near infrared and red light is then absorbed by cells, where it stimulates cellular mitochondria, and triggers the increased production of adenosine triphosphate (ATP). This increase in ATP creates higher functioning cells that stimulate a human body's natural processes. Red light and near infrared light therapy results in benefits such as decreased inflammation, increases in blood flow, increases in collagen production, reduced effects of oxidative stress, and an increased ability to repair and restore damaged cells and tissue. The present invention provides a method which creates a synergistic effect between PEMF therapy and red light and near infrared light therapies.

In various embodiments of the invention, the LED light therapy bed is comprised essentially of an outer body, a LED light array and a controller. In various embodiments of the invention, the LED light therapy bed's outer body is of a clamshell design having a cover unit 5 and a bed unit 6 which connect and move through a hinge mechanism. In embodiments of the invention, a user may lay in the LED light therapy bed and then pull the cover unit downward so as to surround the user with LED light units. Red and infrared LED arrays are located on the inside surface of the cover unit 5 and the bed unit 6 so as to provide thorough irradiation of a user 1.

Persons having skill in the art will readily appreciate that the number of LED units may vary. Furthermore, persons having skill in the art will readily appreciate that combinations of LED units may be used to emit different wavelengths of light. Moreover, power output of the LED light array may vary to produce more or less light upon demand. In embodiments of the invention, the LED light chamber includes a cooling system.

Because of the amount of infrared radiation associated with the LED light therapy bed, the LED light therapy bed 4 includes a cooling system located in both the cover unit 5 and the bed unit 6. Such a cooling system is comprised of radiator fins integrated into the trays which house the LED lights. The heat generated by the LEDs is dissipated into the fins. A fan system carries fresh air through intake vents across the fins, pulling heat off of them, and lowering the ambient temperature of the LED's, the chamber, and the electrical components inside the chamber. There is a direct correlation between the temperature of LED lights and their power output and lifespan. The cooler the temperature, the brighter the LED's are, and the longer they will last. The resulting effect of this cooling system is that it provides superior cooling for the LED's, compared to traditional fan only methods, and as a result the LED's are able to operate at a superior output power and they have a superior lifespan.

In alternative embodiments of the invention, a pulsed electromagnetic field generating device may be integrated into the LED light therapy bed. In such embodiments, a user 1 may lay in the LED light therapy bed for the first two steps of the method of reducing visceral fat in humans. In such an embodiment, a greater synergistic effect is achieved through the use of both PEMF therapies and red and near infrared therapies.

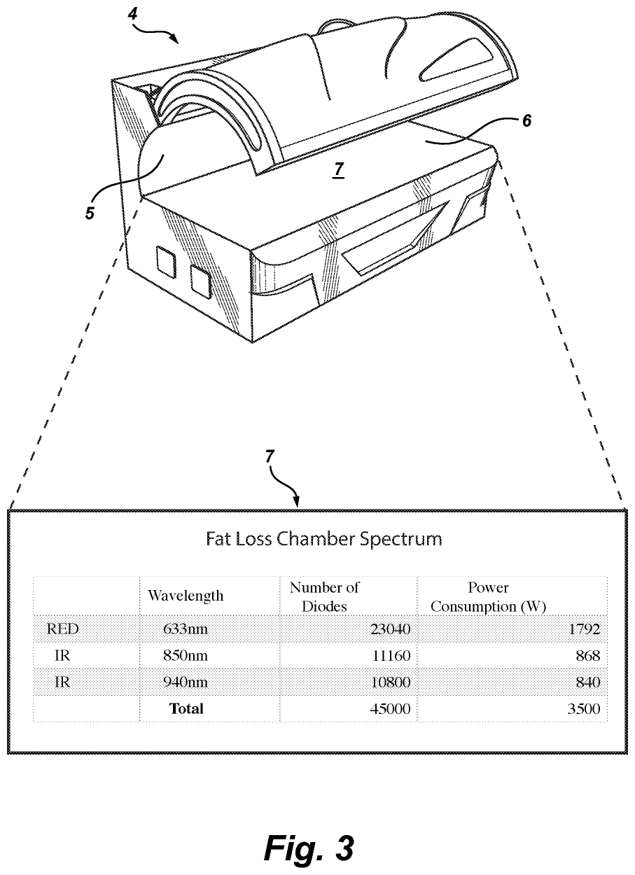

FIG. 3 illustrates a perspective view of an LED light therapy bed 4 and a fat loss chamber spectrum chart 7 in accordance with an embodiment of the invention. The second step of the method for reducing visceral fat in humans involves the user laying in a LED light therapy bed 4. The fat loss chamber spectrum chart 7 recites a preferred embodiment of the LED therapy bed having a LED light array emitting both red and infrared light in a targeted spectrum that emits a wavelength combination that is within a nm range of: ±10 nanometers of 633 nanometers (51.2%), 850 nanometers (24.8%), 940 nanometers (24%). In one embodiment of the invention, the LED light therapy bed uses 45000 LED units, with 23040 units emitting light at the 633 nanometer wavelength, 11160 units emitting light at the 850 nanometer wavelength, and 10800 units emitting light at the 940 nanometer wavelength. Persons having skill in the art will readily appreciate that the number of diodes used, the power consumption and the wavelength of the LED light units may vary according to therapeutic demand.

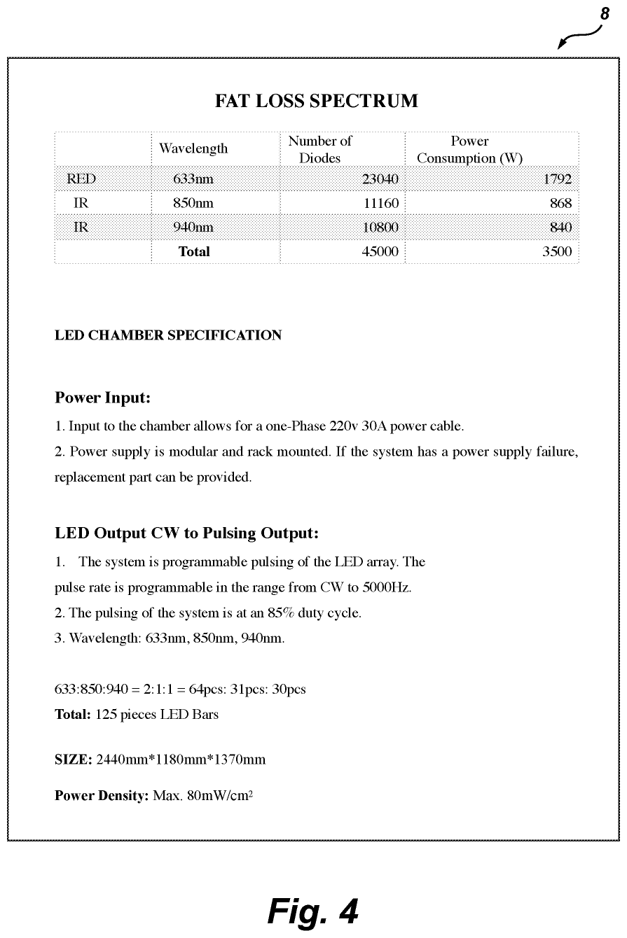

FIG. 4 illustrates a target light spectrum table 8 configurable to the LED therapy bed specifically targeted for the reduction of visceral fat. In embodiments of the invention, the LED light therapy bed is configured to emit chamber spectrum consisting of three different wavelengths of light, the first having a wavelength between 500 and 700 nanometers and an output percentage ranging between 25 and 55 percent, the second having a wavelength between 800 and 900 nanometers and an output percentage ranging between 20 and 30 percent, and the third wavelength between 850 and 1050 nanometers and an output percentage ranging between 20 and 30 percent. In the preferred embodiment of the invention, the LED array of the LED therapy bed produces a chamber spectrum consisting of three different wavelengths of light, the first having a preferred wavelength between ±10 nanometers of 633 nanometers and an output percentage of 51.2 percent, the second having a preferred wavelength between ±10 nanometers of 850 nanometers and an output percentage of 24.8 percent, and the third wavelength a preferred wavelength between ±10 nanometers of 940 nanometers and an output percentage of 24%.

In one embodiment of the invention, the LED light therapy bed has a power input consisting of a one-phase 220 volt, 30 ampere power cable. The power supply may be modular and/or rack mounted.

The LED light therapy bed employ LED units which transmit continuous wave or pulsed outputs programmable to 5000 hertz at 85% of duty. Persons having skill in the art will readily appreciate that a LED light therapy may be programmed through computer processor systems to produce different results depending on therapeutic demand. The number of LED units may vary. However, in one embodiment of the invention, the number of LED units may include LED light bars comprising a 2:1:1 ratio of 633 nanometer wavelength to 850 nanometer wavelength to 940 nanometer wavelength emitting units.

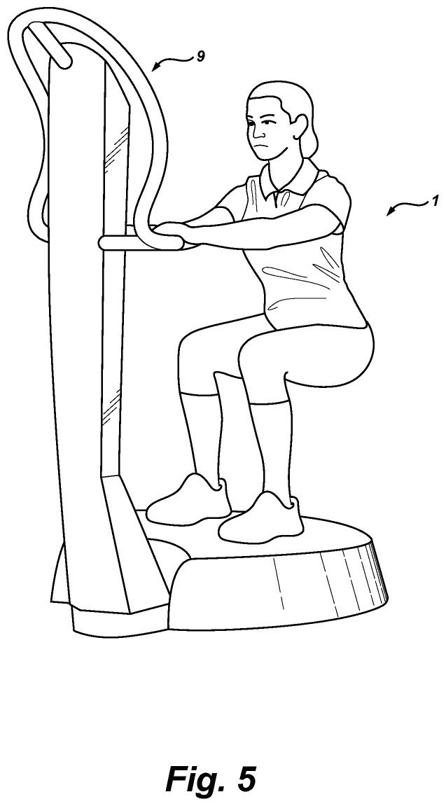

FIG. 5 illustrates a vibration platform machine 9 designed to facilitate increased blood flow throughout the body. A vibration platform machine 9 transmits mechanical vibrational energy to a human body, and by doing so, forces muscles to rapidly contract and relax. The result is muscle motion which utilizes lipids released when the user completes LED light therapy step or phase.

In an embodiment of the invention, a user 1 stands on a vibration platform for 15-30 minutes to facilitate blood flow to body and aid in the reduction and expulsion of visceral fat through natural bodily processes. Results related to increased circulation include, but are not limited to, improved circulation and drainage of the lymph system, which improves the processes by which the body filters and flushes out fat molecules. Persons skilled in the art will understand that a user need not stand on a vibration platform in order to utilize the a vibration platform. In alternative embodiments, the vibration machine may be configured for users to sit on or to lay on depending on user needs.

Immediately following a user standing on a vibration platform The method of claim 8 wherein the user engages in light exercise for a recommended period of time immediately following the step of standing on a vibration platform machine for a preferred length of time. A user may engage in moderate pace walking, light treadmill activity, light bicycle riding or easy swimming. The purpose of such light exercise is to allow for any released lipids, fatty acids and triglycerides to be metabolized by the body. Performed as directed, such a method will result in the loss of visceral fat in a human body.

All the features disclosed in this specification, including any accompanying abstract and drawings, may be replaced by alternative features serving the same, equivalent or similar purpose, unless expressly stated otherwise. Persons skilled in the art will understand that there are multiple means of implementing such a method for reducing visceral body fat involving the steps of a user utilizing a pulsed electromagnetic field generating device for a recommended period of time; the user then laying in an LED therapy bed with a light chamber having an LED array configured to emit multiple wavelengths of light at differing output percentages, the LED array of the LED therapy bed producing a chamber spectrum combination of at least three different light wavelengths for a preferred length of time; and the user then utilizing a vibration platform for a preferred length of time. Thus, unless expressly stated otherwise, each feature disclosed is one example only of a generic series of equivalent or similar features.

Having fully described at least one embodiment of the method for reducing visceral body fat in humans, other equivalent or alternative methods of implementing the method for reducing visceral body fat in humans according to the present invention will be apparent to those skilled in the art. Various aspects of the invention have been described above by way of illustration, and the specific embodiments disclosed are not intended to limit the invention to the particular forms disclosed. The particular implementation of the method for reducing visceral body fat in humans may vary depending upon the particular context or application. By way of example, and not limitation, the method for reducing visceral body fat in humans described in the foregoing was principally directed visceral body fat loss techniques. However, similar techniques may instead be applied to other fat loss and exercise regimens which implementations of the present invention are contemplated as within the scope of the present invention. Such possibilities include, but are not limited to, the use of a greater number of wavelengths of red and/or infrared light. Further alternative embodiments of the present invention may include different types of pulsed electromagnetic field devices, LED light therapy beds and vibration platforms. Further embodiments of the invention may involve the use of detoxification devices, supplemental oxygen therapy, and the use of diagnostic tools in conjunction with the above recited devices. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Although specific features of the method for reducing visceral body fat in humans are shown in some drawings and not others, persons skilled in the art will understand that this is for convenience. Each feature may be combined with any or all of the other features in accordance with the invention. The words “including,” “comprising,” “involving,” “having,” and “with” as used herein are to be interpreted broadly and comprehensively and are not limited to any physical interconnection. Claim elements and steps herein may have been numbered and/or lettered solely as an aid in readability and understanding. Any such numbering and lettering in itself is not intended to and should not be taken to indicate the ordering of elements and/or steps in the claims to be added at a later date.

Any amendment presented during the prosecution of the application for this patent is not a disclaimer of any claim element presented in the description or claims to be filed. Persons skilled in the art cannot reasonably be expected to draft a claim that would literally encompass each and every equivalent.

What is claimed is:

1. A method for reducing visceral body fat in a human user involving the steps of:
a. The user laying on or in a pulsed electromagnetic field generating device and being exposed to pulsed electromagnetic radiation for a recommended period of time to stimulate muscles, increase microcirculation, and prepare the user for red and infrared light therapy;
b. the user laying in a clamshell shaped LED therapy bed with a light chamber having an LED array configured to emit a chamber spectrum consisting of multiple wavelengths of light at differing output percentages so as to open pores on the surface of visceral fat cells and release lipids and fatty acids upon exposure to said multiple of light for a recommended period of time, and;
c. the user standing on a vibration platform for a preferred length of time to facilitate increased blood flow throughout the user's body.

2. The method of claim 1 wherein the user engages in light exercise for a recommended period of time immediately following the step of standing on a vibration platform machine for a preferred length of time.

3. The method of claim 1 wherein the recommended period of time for the user laying on the pulsed electromagnetic field device is between eight and twenty minutes.

4. The method of claim 1 wherein the LED array of the clamshell shaped LED therapy bed produces a chamber spectrum consisting of three different wavelengths of light, the first having a wavelength between 500 and 700 nanometers, the second having a wavelength between 800 and 900 nanometers, and the third wavelength between 850 and 1050 nanometers.

5. The method of claim 1 wherein the LED array of the clamshell shaped LED therapy bed produces a chamber spectrum consisting of three different wavelengths of light, the first having a wavelength between 500 and 700 nanometers and an output percentage ranging between 25 and 55 percent, the second having a wavelength between 800 and 900 nanometers and an output percentage ranging between 20 and 30 percent, and the third wavelength between 850 and 1050 nanometers and an output percentage ranging between 20 and 30 percent.

6. The method of claim 1 wherein the recommended period of time for the user laying in the clamshell shaped LED therapy bed is between ten and twenty minutes.

7. The method of claim 1 wherein the recommended period of time for the said human user standing on the vibration platform is between 15 and 30 minutes.

8. A method for reducing visceral body fat in a human user involving the steps of:
a. The user laying on or in a pulsed electromagnetic field generating device and being exposed to pulsed electromagnetic radiation to prepare the user's body for red and infrared light therapy by way of stimulating muscles, improving circulation and microcirculation throughout the user's body for a recommended period of time;
b. immediately following the user laying on or in a pulsed electromagnetic field generating device for a recommended period of time, the user laying in a clamshell shaped LED therapy bed with a light chamber having an LED array comprising 45000 LED units configured to emit a chamber spectrum consisting of multiple wavelengths of light at differing output percentages so as to open pores on the surface of visceral fat cells and release lipids and fatty acids upon exposure to said multiple wavelengths of light for a recommended period of time, and;
c. immediately following the user laying in a clamshell shaped LED therapy bed with a light chamber having an LED array comprising 45000 LED units configured to emit a chamber spectrum consisting of multiple wavelengths of light at differing output percentages, the user standing on a vibration platform machine for a preferred length of time to facilitate increased blood flow throughout the body and to utilize lipids and fatty acids released after laying in the said LED light therapy bed.

9. The method of claim 8 wherein the user engages in light exercise for a recommended period of time immediately following the step of standing on a vibration platform machine for a preferred length of time.

10. The method of claim 8 wherein the recommended period of time for the user laying on the pulsed electromagnetic field device is between eight and twenty minutes.

11. The method of claim 8 wherein the LED array of the clamshell shaped LED therapy bed produces a chamber spectrum consisting of three different wavelengths of light, the first having a wavelength between 500 and 700 nanometers, the second having a wavelength between 800 and 900 nanometers, and the third wavelength between 850 and 1050 nanometers.

12. The method of claim 8 wherein the LED array of the clamshell shaped LED therapy bed produces a chamber spectrum consisting of three different wavelengths of light, the first having a wavelength between 500 and 700 nanometers and an output percentage ranging between 25 and 55 percent, the second having a wavelength between 800 and 900 nanometers and an output percentage ranging between 20 and 30 percent, and the third wavelength between 850 and 1050 nanometers and an output percentage ranging between 20 and 30 percent.

13. The method of claim 8 wherein the LED array of the clamshell shaped LED therapy bed produces a chamber spectrum consisting of three different wavelengths of light, the first having a preferred wavelength between ±10 nanometers of 633 nanometers and an output percentage of 51.2 percent, the second having a preferred wavelength between ±10 nanometers of 850 nanometers and an output percentage of 24.8 percent, and the third wavelength a preferred wavelength between ±10 nanometers of 940 nanometers and an output percentage of 24%.

14. The method of claim 8 wherein the recommended period of time for the user laying in the clamshell shaped LED therapy bed is between ten and twenty minutes.

15. The method of claim 8 wherein the recommended period of time for the user standing on the vibration platform is between 15 and 30 minutes.

16. A method of reducing visceral body fat in a human user involving the steps of:
a. The user laying on or in a pulsed electromagnetic field generating device and being exposed to pulsed electromagnetic radiation to prepare the user's body for red and infrared light therapy by way of improving circulation and microcirculation throughout the user's body for a recommended period of time, the said pulsed electromagnetic field generating device being integrated into a clamshell shaped LED therapy bed with a light chamber having an LED array comprising 45000 LED units configured to emit a chamber spectrum consisting of multiple wavelengths of light at differing output percentages so as to open pores on the surface of visceral fat cells and release lipids and fatty acids upon exposure to said multiple wavelengths of light for a recommended period of time, and;
b. The user standing on a vibration platform machine for a preferred length of time to facilitate increased blood flow throughout the body and to utilize lipids and fatty acids released after laying in the said LED light therapy bed.

17. The method of claim 16 wherein the user engages in light exercise for a recommended period of time immediately following the step of standing on a vibration platform machine for a preferred length of time.

18. The method of claim 16 wherein the recommended period of time for the user laying on the pulsed electromagnetic field device is between eight and twenty minutes.

19. The method of claim 16 wherein the LED array of the clamshell shaped LED therapy bed produces a chamber spectrum consisting of three different wavelengths of light, the first having a wavelength between 500 and 700 nanometers and an output percentage ranging between 25 and 55 percent, the second having a wavelength between 800 and 900 nanometers and an output percentage ranging between 20 and 30 percent, and the third wavelength between 850 and 1050 nanometers and an output percentage ranging between 20 and 30 percent.

20. The method of claim 16 wherein the LED array of the clamshell shaped LED therapy bed produces a chamber spectrum consisting of three different wavelengths of light, the first having a preferred wavelength between ±10 nanometers of 633 nanometers and an output percentage of 51.2 percent, the second having a preferred wavelength between ±10 nanometers of 850 nanometers and an output percentage of 24.8 percent, and the third wavelength a preferred wavelength between ±10 nanometers of 940 nanometers and an output percentage of 24%.
